(19) United States
(12) Patent Application Publication
(10) Pub. No.: US 2023/0018153 A1
(43) Pub. Date: Jan. 19, 2023

(54) ENCLOSURES FOR RAISING COCOON-FORMING INSECTS

(71) Applicant: NED A. BRUNS, Elk Grove Village, IL (US)

(72) Inventor: NED A. BRUNS, Elk Grove Village, IL (US)

(21) Appl. No.: 17/374,710

(22) Filed: Jul. 13, 2021

(51) Int. Cl. A01K 67/033; A47B 43/02; A47B 81/00; A47B 47/06

(52) U.S. Cl. CPC A01K 67/033; A47B 43/02; A47B 81/00; A47B 47/06; A47B 2220/0086

(57) ABSTRACT

Enclosures for raising cocoon-forming insects are provided. The enclosures, and methods using those enclosures, provide simpler, safer, and more efficient insect raising and reduce the chances of spreading infectious diseases, for example, Ophryocystis Elektroscirrha, between sequential groups of insects being raised. Typically, a first enclosure and second enclosure provide for the growth of the insects until the cocoon is formed and a place for the cocoons to hang until the insects emerge, respectively.

### CROSS-REFERENCE TO RELATED APPLICATIONS

This application is being filed as a non-provisional patent application.

### BACKGROUND OF THE INVENTION

### Field of the Invention

This disclosure relates to the field of raising insects. Enclosures are provided that allow for simpler, safer, and more efficient insect raising.

### Problem to be Solved

Infections that occur during insect raising can remain associated with the enclosures that are used to house the insects. To raise cocoon-forming insects safely and efficiently, it is helpful to transfer insects that have formed cocoons in a first enclosure to a second enclosure so new insects can be added to the first enclosure. However, the transfer and reuse of the enclosures involved in insect raising can carry the infections through to future rounds of insect raising.

### Description of Prior Art

A number of cocoon-forming insect raising enclosures have been developed and marketed for use at home or for classroom instruction. Conventionally, such enclosures include either a clear plastic container or a housing container in the form of a plastic tent or cardboard box with a thin plastic sheet window. The user is afforded an ongoing view of the developing butterfly, for example, through a wall or clear plastic window engaged with the housing. Many individuals in the United States are trying to help boost the dwindling monarch butterfly population by hand raising monarch caterpillars in their homes.

One piece of prior art, the “Lid Box”<https://mamapapabubba.com/tag/painted-lady-butterflies/> describes a box block with four cardboard walls that is open on two sides (front/back). The product is used to hang the chrysalises. After the newly formed chrysalises have been left alone for 12-24 hours, the lids of the containers housing the monarchs are removed and gently attached to the top/ceiling of the product using a good strong masking tape. The chrysalises hang until the final step of their transformation takes place. The product has no removable cardboard shelf that is situated at the top of the unit resting on precut notches at the top of the enclosure. Additionally, the product lacks the door with creases which allow the door to be partially or fully open when removing butterflies.

Another piece of prior art, the “Plant Box” <https://www.pinterest.com/pin/782289397749736701/> is meant to be used to cover natural plants outside that have caterpillars eating them, or that have chrysalises on them. The product comprises four cardboard walls with no bottom and a removable screen top. The walls have holes cut in them in which screens have been placed. There are no shelves. The product keeps predators away from the caterpillars on the plants. The product lacks knock-in shelf supports for even one shelf. The product is a single box and lacks the second box of the presently disclosed invention (where the chrysalises are moved to). The product lacks a front that can be opened to different compartments.

Another piece of prior art, the “Wood Cage”<https://homesteadandchill.com/all-about-raising-monarch-butterflies/> describes a wooden frame with a solid wood top and bottom, three screened sides for the sides and back, and a screened door that is hinged and able to be latched. There is a place for a stick to be laid across about ¾ of the way up the product to which chrysalises are tied. The product is used for the last stage of butterfly development, when chrysalises hang until the butterflies emerge. The product does not have a removeable shelf. Instead of the simple method of moving an entire shelf of chrysalises to a new box, the Wood Cage details a time-intensive method of moving chrysalises using string and thread. In contrast, the product would likely have to be thoroughly cleaned frequently to prevent OE infection. The product has a door that can be opened, but does not have the ability to open individual compartments of the cage.

An additional problem of the above enclosures is that some use wood, vinyl, plastic, and metal. Such materials, when employed, are much more expensive than cardboard. Further, the use of these materials would be much heavier in weight. A cardboard construction makes them lighter for handling as well as shipping.

Thus, there is a continuing need for improved cocoon-forming insect raising enclosures. Such enclosures need to be relatively sturdy, yet permit easy observation of the ongoing natural process through the provision of a viewing window or wall. Such an improved device, should provide knock-in shelf supports to add shelves in different places for different boxes, the use of multiple shelves, compartmentalization of the chrysalises, a moveable shelf to transfer chrysalises from one box to another, or a door that can be opened to individual compartments of a box. Finally, such an enclosure should employ an inexpensive cardboard material to render the eventual cost of the device sufficiently inexpensive to encourage widespread use.

### STATEMENT OF THE OBJECTS OF THE INVENTION

It is the purpose of the current invention to provide enclosures for raising cocoon-forming insects that allow the raising of more insects in the same space and the reduction of infection outbreaks.

### SUMMARY OF THE INVENTION

The invention includes, in some embodiments, an enclosure for raising cocoon-forming insects, and in illustrative embodiments raising monarch butterflies, comprising: a left side, a right side, a front, a back, a top, and a bottom; the left side, the right side, and the front each comprising a screen; the left side and the right side comprising at least one set of corresponding knock-in shelf supports dividing the interior of the enclosure into at least two compartments, the at least two compartments comprising a top compartment and a bottom compartment. The knock-in shelf supports form corresponding sets of knock-in shelf supports and are located similar distances from the bottom of the enclosure such that the shelves are level or close to level. The sets of corresponding knock-in shelf supports, when knocked-in on the left side and the right side, support a shelf, which in illustrative embodiments is a cardboard shelf.

### BRIEF DESCRIPTION OF THE DRAWINGS

FIG. 1 is a front view of an exemplary first enclosure for raising cocoon-forming insects.

FIG. 2 is a view of the interior of the exemplary first enclosure for raising cocoon-forming insects of FIG. 1 where the front has been removed.

FIG. 3 is a perspective view of the front and right sides of the exemplary first enclosure for raising cocoon-forming insects of FIG. 1 with a partially-opened front.

FIG. 4 is a perspective view of the front and left sides of the exemplary first enclosure for raising cocoon-forming insects of FIG. 1 with a partially-opened front.

FIG. 5 is a front view of an exemplary second enclosure for raising cocoon-forming insects.

FIG. 6 is a view of the open front of the exemplary second enclosure for raising cocoon-forming insects of FIG. 5.

FIG. 7 is a perspective view of the front and right sides of the exemplary second enclosure for raising cocoon-forming insects of FIG. 5 with a partially-opened front.

FIG. 8 is a perspective view of the front and left sides of the exemplary first enclosure for raising cocoon-forming insects of FIG. 1 with a partially-opened front.

### DETAILED DESCRIPTION

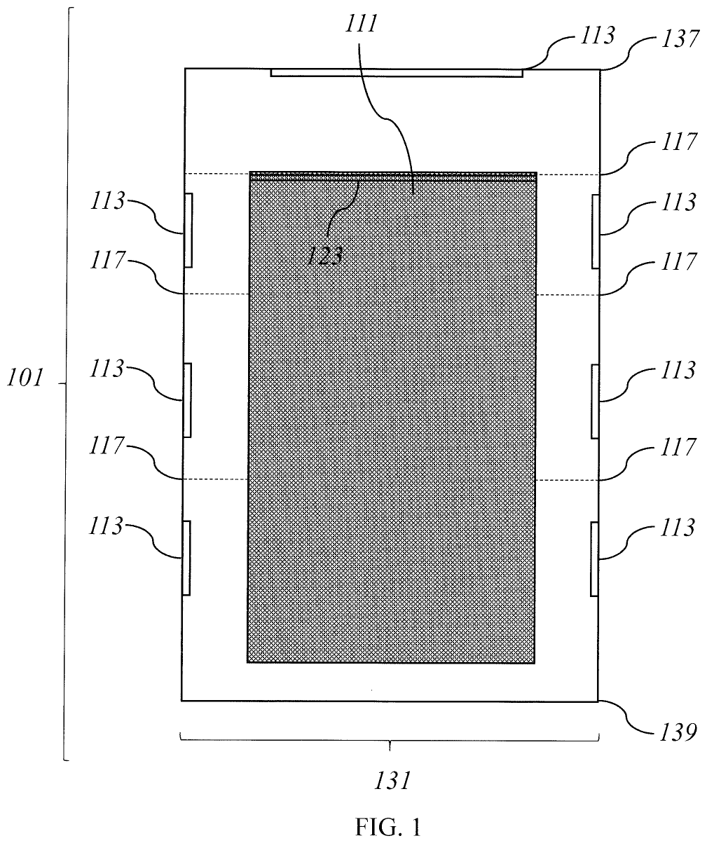

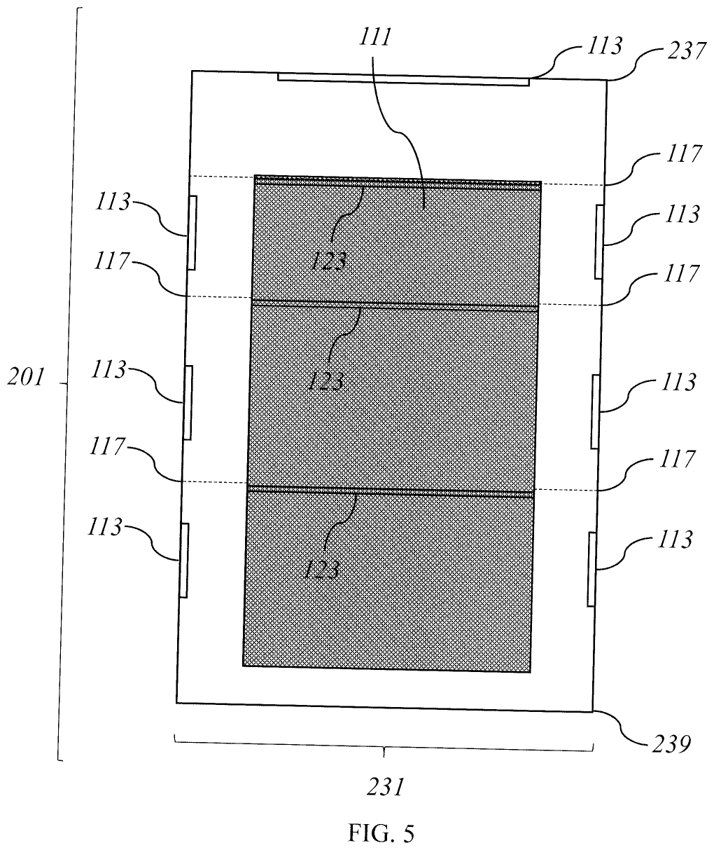

The enclosures and methods disclosed herein achieve the above-mentioned goals and objects, through the provision of enclosures which allow for simpler, safer, and more efficient insect raising while reducing chances of spreading infectious diseases, for example Ophryocystis Elektroscirrha (“OE”), between sequential groups of insects being raised. Generally, a first enclosure embodiment (FIGS. 1-4) provides for the growth of the insects until the cocoon, e.g., chrysalis, is formed and a second enclosure embodiment (FIGS. 5-8) allows the cocoons to hang until the insects emerge. For example, a first group of cocoon-forming insects are placed in the first compartment with a first shelf at the top. The cocoon-forming insects are allowed to form cocoons on, e.g., attach their chrysalises to, the first shelf, and the first shelf is moved to a shelf support, such as knock-in shelf supports, at the top of a second compartment. A second group of cocoon-forming insects are placed in the first compartment. The second group of cocoon-forming insects are allowed to form cocoons on a new, second shelf sitting at the top, and the second shelf is moved to the second compartment below the first shelf. As the first group of insects emerge from their cocoons, they are released through the front of the second compartment.

Once all insects from the first group have emerged from their cocoons, the first shelf is removed from the second compartment, and the second shelf is moved to the top. After removal, the first shelf is optionally discarded or washed. During this time, another group of insects can be added to the first compartment with a new, third shelf at the top, and once cocoons are formed on it, the third shelf is transferred into the second compartment below the second shelf. Such rotations and incubations in the different compartments allow the cocoon-forming insects to be efficiently raised, even in places with limited space. The transfer of the shelves from the first compartment to the second compartment reduces the chances of an infection remaining in the first compartment. This allows the second, third, and further groups to grow healthily. Similarly, the removal of shelves from the second compartment after the insects emerge from their cocoons prevents any holdover of infection. Discarding the shelves or washing and disinfecting them similarly reduces the chances of any infection being reintroduced into the compartments.

In one embodiment, a removable cardboard shelf is situated at the top of the first compartment resting on four knocked-in shelf supports at the top of the compartment. The insect, and in illustrative embodiments the caterpillar, forms a cocoon on the bottom of this removable cardboard shelf. A second cardboard compartment is set up by knocking in the twelve knock-in shelf supports on the side walls of the enclosure. These knock-in shelf supports support removable cardboard shelves on which cocoons are attached. These shelves remain in the second compartment until the insects emerge. Paper towels can be placed under the cocoons to catch any moisture or fluids that may collect while the insects, and in illustrative embodiments the butterflies, emerge from the cocoons. Shelves can be rotated to the top of the unit to make removal of insects easier. Once all the insects have been removed from any one shelf, the shelf can be discarded and replaced with a new shelf with attached cocoons from the first compartment. Creases in the pivoted front door allow the door to be partially or fully open when removing the insects.

In one embodiment, provided herein is an enclosure for raising cocoon-forming insects, comprising:

a left side, a right side, a front, a back, a top, and a bottom;

the left side, the right side, the front, and back each comprising of a screen;

the left side and the right side comprising at least one set of corresponding knock-in shelf supports dividing the interior of the enclosure into at least two compartments, the at least two compartments comprising a top compartment and a bottom compartment;

each set of corresponding knock-in shelf supports located on the left side of the enclosure and the right side of the enclosure similar distances from the bottom of the enclosure; and

the sets of corresponding knock-in shelf supports, when knocked-in on the left side and the right side, capable of supporting a shelf.

In another embodiment, provided herein are a plurality of flaps on a cardboard door, the plurality of flaps folded into the front of the upright cardboard box;

at least four flap receptors secured to upright front of said cardboard box, each of said flap receptors having area or slots for receiving at least one of the plurality of flaps on said cardboard door;

at least three creases in said cardboard door that allow the cardboard door to be partially or fully open when removing butterflies;

at least twelve perforations on the side walls of said cardboard box, the at least twelve perforations serving as shelf holders; and,

at least three removable cardboard shelves supported by the shelf holders.

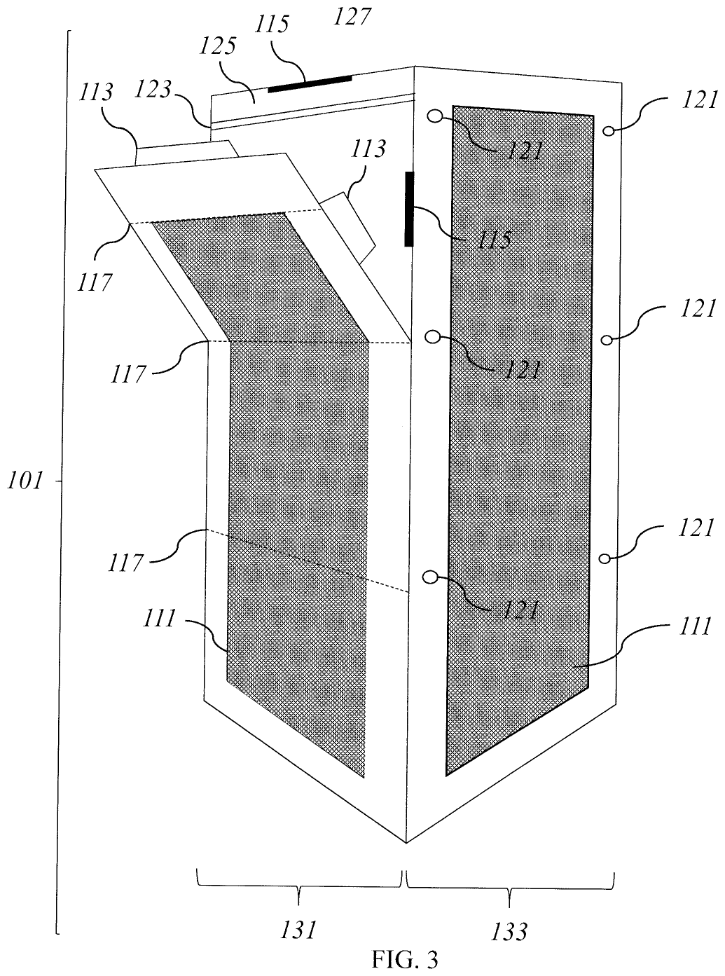

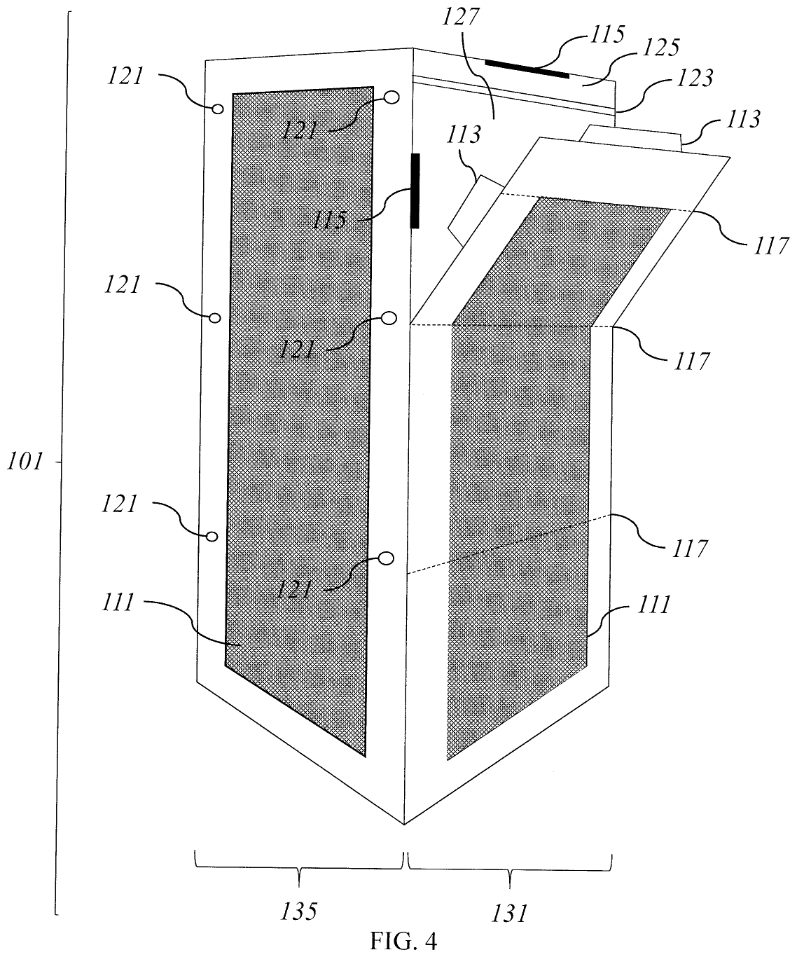

Various views of exemplary embodiments of enclosures for raising cocoon-forming insects are disclosed, including first enclosure (101) (FIGS. 1-4) and second enclosure (201) (FIGS. 5-8). The front view of a first front (131) of a first enclosure (101) is shown in FIG. 1. The first front (131) includes a screen (111) and one shelf (123) visible through the screen, near the first top (137) of the first enclosure (101). Beneath the first top (137) is the first bottom (139) of the first enclosure (101). Along the sides and top the first front (131) are door flaps (113) that connect the first front to the first right side (133) and the first left side (135) and first top (137)(FIGS. 3-4). The first front (131) also has creases (117) to allow access to the interior of the first enclosure (101) without opening the entire first front (131).

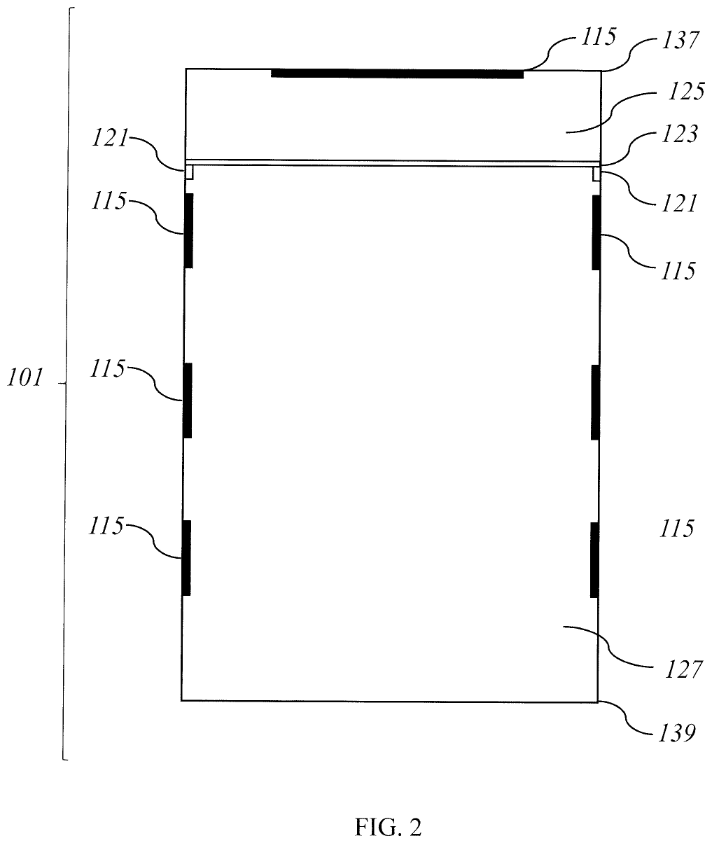

The interior of first enclosure (101), with the first front (131) not seen, is shown from the front in FIG. 2. This interior view shows the one shelf (123) supported by the knocked-in shelf supports (121). Between the shelf (123) and the first top (137) is the top compartment (125) and between the shelf (123) and the first bottom (139) is the bottom section (127). The flap receptors (115) along the sides and top are where the door flaps (113) in FIG. 1 are inserted.

A perspective view of the first front (131) and first right side (133) of a first enclosure (101) are shown in FIG. 3. The shelf (123), supported by the knocked-in knock in shelf supports (121), divides the interior of the first enclosure (101) into the top compartment (125) and the bottom compartment (127) (FIG. 2). The first front (131) and first right side (133) both include a screen (11). The first front (131) is opened at two of the creases (117), with the door flap (113) and flap receptor (115) visible.

A perspective view of the first front (131) and first left side (135) of the first enclosure (101) are shown in FIG. 4. The shelf (123), supported by the knocked-in knock in shelf supports (121), divides the interior of the first enclosure (101) into the top compartment (125) and the bottom compartment (127). The first front (131) and first left side (135) both include a screen (111). The first front (131) is opened at two of the creases (117), with the door flap (113) and flap receptor (115) visible.

The front view of a second front (231) of a second enclosure (201) is shown in FIG. 5. The second front (231) includes a screen (111) and three shelves (123) visible through the screen; one shelf (123) closest to the second top (237) of the second enclosure (201), one shelf (123) closest to the second bottom (239) of the second enclosure (201), and one shelf (123) in between the other two shelves (123). Along the sides of the second front (231) are the door flaps (113) that connect the second front to the second right side (233) and the second left side (235). The second front (231) also has creases (117) to allow access to the interior of the second enclosure (201) without removing or opening the entire second front (231).

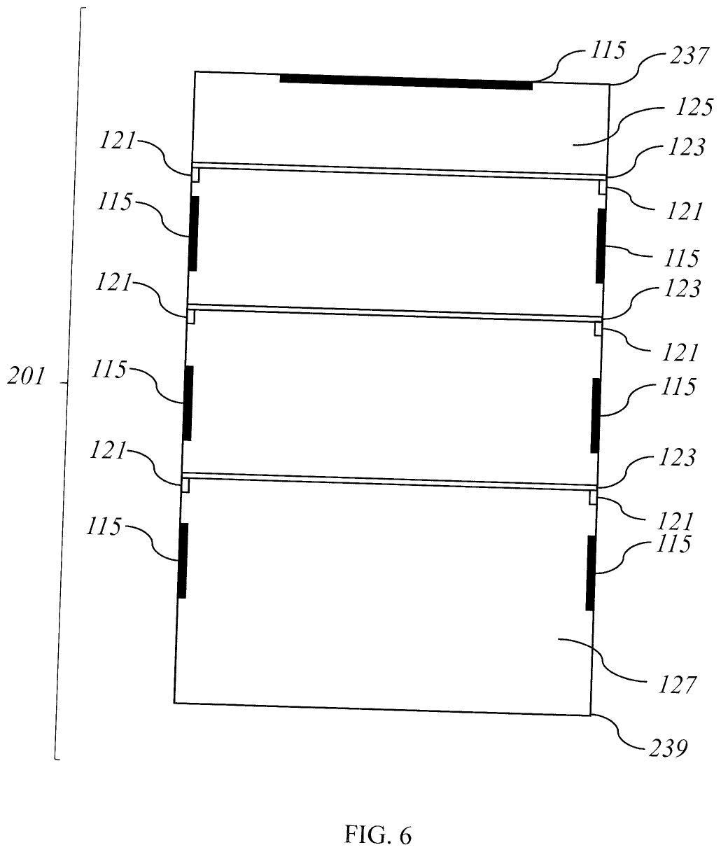

The interior of the second enclosure (201), with the second front (231) not seen, is shown from the front in ______ FIG. 6. This interior view shows three shelves (123), thus the second enclosure has two more shelves (123) than the first enclosure (FIG. 2). Between the shelf (123) closest to the second top (237) and the second top (237) is the top compartment (125) and between the shelf (123) closest to the second bottom (239) and the second bottom (239) is the bottom compartment (127), with one shelf (123) in between the other two shelves (123). The flap receptors (115) along the sides and top are where the door flaps (113) insert. In some embodiments, the shelves (123) are transferred from the knock-in shelf supports (121) closer to the second bottom (239) to the next set of knock-in shelf supports (121) closer to the second top (237) during the methods, for example after insects emerge from a shelf (123) located closest to the second top (237). By transferring the shelves (123) to the top as the insects emerge from their cocoons, the next group of insects will be in the top compartment as they emerge from their cocoons. The pivoted front door can be partially opened such that only the top compartment (125) and below second top shelf (123) area is accessible and the insects can leave the second enclosure (201) as they emerge from their cocoons, without providing access to the lower compartments.

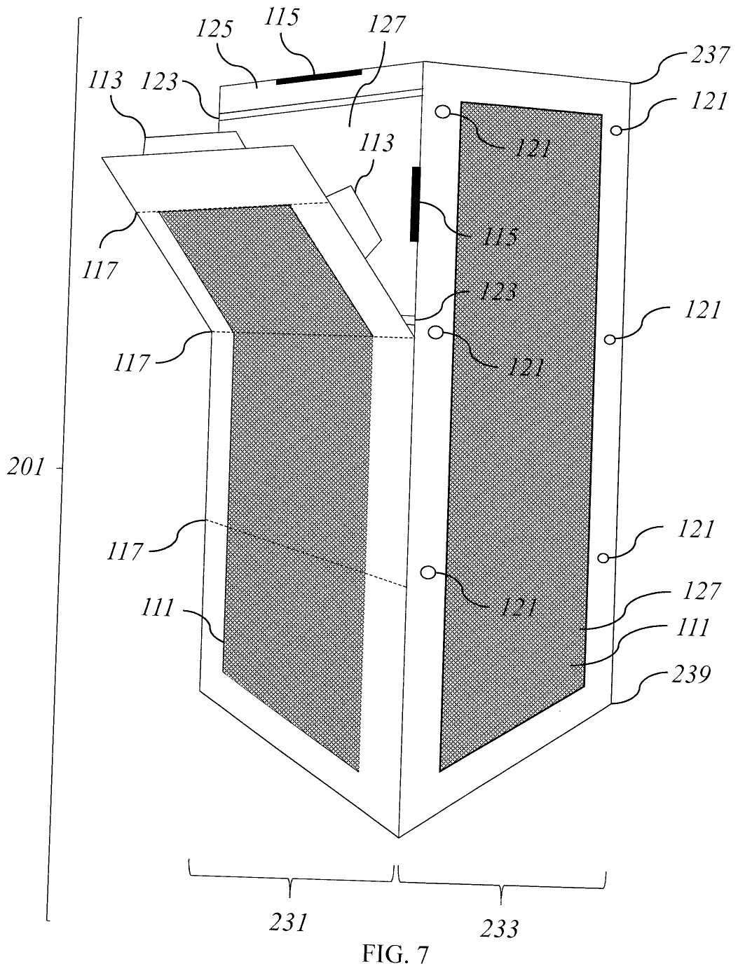

A perspective view of the second front (231) and second right side (233) of a second enclosure (201) is shown in FIG. 7. The shelves (123) are supported by the knocked-in knock in shelf supports (121). Between the shelf closest to the second top (237) and the second top (237) is the top compartment (125). The bottom compartments are not visible behind the second front (231). The second front (231) and second right side (233) both include a screen (1l1). The second front (231) is opened at two of the creases (117), with the door flap (113) and flap receptor (115) visible.

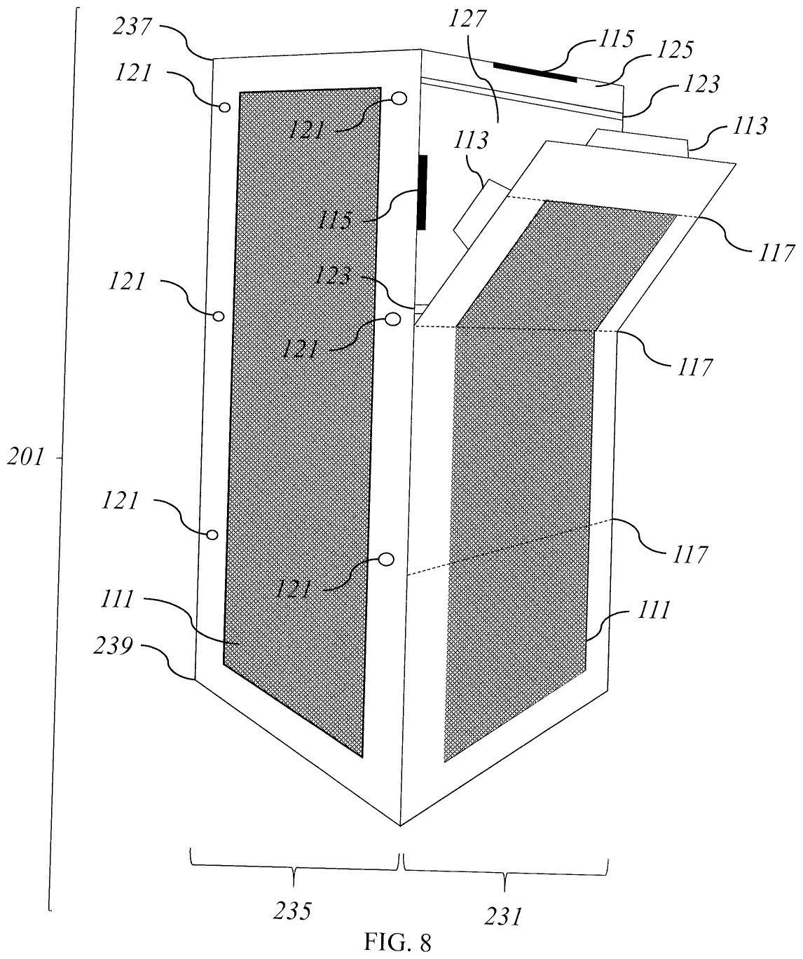

A perspective view of the second front (231) and second left side (235) of a second enclosure (201) is shown in FIG. 8. The shelves (123) are supported by the knocked-in knock in shelf supports (121). Between the shelf closest to the second top (237) and the second top (237) is the top compartment (125). The bottom compartments are not visible behind the second front (231). The second front (231) and second left side (235) both include a screen (111). The second front (231) is opened at two of the creases (117), with the door flap (113) and flap receptor (115) visible.

In some embodiments, the overall enclosure comprises wood, vinyl, plastic, metal, cardboard, or paper. In illustrative embodiments, the enclosure comprises cardboard. In some embodiments, the shelves comprise wood, vinyl, plastic, metal, cardboard, or paper. In illustrative embodiments, the shelves comprise cardboard. The inexpensive and recyclable nature of cardboard allows it to be discarded on a regular basis (for example, season to season or group to group) to reduce the chances of OE infection spreading.

To ease access and selective opening of the enclosure, the front of the enclosure can include creases or hinges to allow the front to be partially opened such that one interior compartment is accessible while keeping the other compartments closed (FIGS. 3-4 and 7-8). Thus, in some embodiments, the front of the enclosure comprises at least one, two, three, or four creases or hinges. In illustrative embodiments, the front comprises cardboard and at least one, two, three, or four creases.

In some embodiments, the knock-in shelf supports comprise pegs, tabs, or flaps.

In some embodiments, a second shelf rests on a second set of corresponding knock-in shelf supports that have been knocked in, and a third shelf rests on a third set of corresponding knock-in shelf supports that have been knocked in.

In some embodiments, the front is removable. In illustrative embodiments, the front is not removable. In illustrative embodiments, at least one compartment is accessible from the front without accessing any other compartments, for example, by opening the front to the second crease from the top of door.

In some embodiments, the cocoon-raising enclosure is constructed using a hot adhesive (in illustrative embodiments, hot glue), tape, staples, safety pins, screws, nails, nuts, or bolts. In illustrative embodiments, the cocoon-raising enclosure is constructed using tape. In some embodiments, the screens of the cocoon-raising enclosure are attached using hot glue, tape, staples, or safety pins. In illustrative embodiments, the screens of the cocoon-raising enclosure are attached using hot glue.

In some embodiments, the first shelf is removeable. In illustrative embodiments, the first shelf, the second shelf, and the third shelf are removeable.

In some embodiments, the first shelf is situated at the top of the cocoon-raising enclosure. In some embodiments, any shelf can be transferred to an empty set of corresponding knocked-in enclosures after another shelf is removed. In illustrative embodiments, the second shelf and the third shelf can be transferred to be the first and second shelves of the enclosure, respectively, after the first shelf is removed.

In some embodiments, the back or top comprise a screen.

In some embodiments, the cocoon-raising enclosure is made of wood. In illustrative embodiments, the cocoon-raising enclosure is made of balsa wood.

In some embodiments, the cocoon-forming insects are butterflies, moths, beetles, flies, ants, bees, wasps, or fleas. In illustrative embodiments, the cocoon-forming insects are butterflies. In further illustrative embodiments, the butterflies are Monarch butterflies. In some embodiments, the cocoon-forming insects are members of the Lepidoptera, Neuroptera, Mecoptera, Trichoptera, Strepsiptera, Coleoptera, Hymenoptera, Diptera, Siphonaptera, or Nematocera families. In illustrative embodiments, the cocoon-forming insects are members of the Lepidoptera family.

In some embodiments, the sides, front, back, top, and bottom can be made in various combinations and then assembled, for example using glue or tape, to form the cocoon-raising enclosure. In some embodiments, any or all of the sides, front, back, top, or bottom can be made separately. In illustrative embodiments, the sides, top, and bottom are made as one piece and the front and back are each made as separate pieces, and then the three pieces are assembled to form the cocoon-raising enclosure.

In another aspect, provided herein is a method of raising cocoon-forming insects, comprising the steps of—

a) placing a plurality of cocoon-forming insects in a bottom compartment (127) of a first enclosure (101), wherein the plurality of cocoon-forming insects are at a life stage before forming a cocoon in a first compartment of the first enclosure;

b) feeding the plurality of cocoon-forming insects;

c) waiting for a quantity of the cocoon-forming insects to form a cocoon on the shelf (123);

d) transferring the shelf (123) comprising the cocoons to a second compartment in a second enclosure (201); and

e) waiting for at least some of the cocoon-forming insects that formed a cocoon to emerge from the cocoons.

To begin the method, cocoon-forming insects can be collected from their known environments or farmed using methods known in the art. For example, Monarch caterpillars feed exclusively on milkweed, such that eggs and caterpillars can be found on this plant when Monarch caterpillars are present in the environment. Once collected, the cocoon-forming insects can be placed inside a plastic vial or sealed cup with a lid and typically cushioned with paper towels. If the cocoon-forming insects are in an earlier stage of development, they can be fed and cared for in a suitable container until they reach the developmental stage appropriate for the method, typically the last developmental stage before the insect forms a cocoon. Once the appropriate developmental stage is reached, the cocoon-forming insects are transferred into the enclosures by opening the plastic tray or the sealed cup and placing the cocoon-forming insects (which are in the plastic tray or cup) into the enclosures.

The enclosures are configured to raise and protect the cocoon-forming insects during the last three stages of their development—fifth instar caterpillar, chrysalis, and butterfly—from natural events. and predators. Such events may include wind, direct sunlight, changes in temperature, and natural weather events that may harm the cocoon-forming insects. Natural predators may include animals and insects that may harm the cocoon-forming insects and their development. This enables the cocoon-forming insects to develop properly without external disturbances.

In the first enclosure embodiment of FIGS. 1-4, the first left side (135), the first right side (133), and the first front (131), each comprise a screen such that a user can view the metamorphosis of the cocoon-forming insects.

The first enclosure embodiment is used to house the cocoon-forming insects during the fifth instar stage. A removable shelf is situated at the top of the first enclosure embodiment resting on four knocked-in shelf supports at the top of the structure, where the cocoons will be attached. When the cocoon-forming insects are ready to form a cocoon, they will spin a small silk button at the bottom of the removable shelf. The cocoon-forming insects crawl to the bottom of the removable shelf in the first enclosure embodiment, attach themselves with silken thread to the bottom, hang upside down, and assume a J-shaped position. The cocoon-forming insects will expand until their skin splits to reveal a green cuticle. This cuticle hardens and the cocoon-forming insects become a chrysalis.

Using the second enclosure embodiment of FIGS. 5-8, the second compartment is prepared by knocking in twelve shelf supports on the sides of the second enclosure. These precut knocked-in shelf supports can hold three removeable shelves transferred from the first compartment. The three removable shelves will each have cocoons or chrysalis attached to them when the shelves are transferred from the first enclosure. These shelves remain in the second compartment until the insects emerge from their cocoons, typically with a predictable time frame. For example, Monarch butterflies, will typically emerge from their cocoons within 10-14 days if the chrysalis are kept at room temperature. Some moisture may be released during the insects emergence, and paper towels can be placed under the cocoons to catch it. Insects recently emerged from a cocoon are soft and their wings wrinkled and pliable. The wings are also generally damp from their restricted stay within the chrysalis. Their abdomens will be large, and fluid is pumped from the abdomen into the wings, until the wings are fully expanded and become stiff. It may take 1-2 hours for the insects' wings to harden sufficiently for flight.

Shelves can be rotated to the top of the unit to make removal of the butterflies easier. The first enclosure only holds one shelf and the second enclosure holds three shelves, so several rounds of cocoon formation can be performed within the first enclosure and all stored in the second enclosure. The round can be performed sequentially within one first enclosure or in parallel in multiple first enclosures. In either case, each shelf is transferred to the second enclosure when a sufficient number of cocoons have attached to the underside of the shelf. In some embodiments, the shelves can be arranged in the second enclosure such that the shelf having cocoons that are closest to emergence are the top shelf. In such embodiments, once the insects emerge from the cocoons attached to the top shelf, and the top shelf is removed and discarded, the shelf immediately below the top shelf (second from the top) is moved to the top shelf supports, the shelf third from the top is moved to the shelf supports second from the top, and so on. A new shelf with cocoons attached can then be placed on the bottom shelf supports. Once all butterflies have been removed from any shelf it can be discarded and replaced with a newly loaded shelf from the first compartment.

Once the butterflies have completely emerged and can fly, they are released into the environment by opening the creases in the door of the enclosures.

The removeable shelving should never be reused to prevent transfer of Ophryocystis Elektroscirrha (OE).

In another aspect, provided herein is a method of raising cocoon-forming insects in a first enclosure and a second enclosure, comprising the steps of:

a) placing a plurality of cocoon-forming insects in a bottom compartment (127) of the first enclosure (101), wherein the plurality of cocoon-forming insects are at a life stage before forming a cocoon in a first compartment of the first enclosure;

the first enclosure (101) comprising:
a first left side (135), a first right side (133), a first front (131), a first back, a first top (137), and a first bottom (139);
the first left side (135), the first right side (133), and the first front (131) each comprising a screen (111);
the first left side (135) and the first right side (133) comprising at least one set of corresponding knock-in shelf supports (121) dividing the interior of the first enclosure into at least two compartments, the at least two compartments comprising a top compartment (125) and the bottom compartment (127);
each set of corresponding knock-in shelf supports (121) having, on the first left side (135) of the first enclosure (101) and the first right side (133) of the first enclosure (101), similar distances from the first bottom (139) of the first enclosure (101);
the sets of corresponding knock-in shelf supports (121), when knocked-in on the first left side (135) and the first right side (133), capable of supporting a shelf (123);

b) feeding the plurality of cocoon-forming insects;

c) waiting for a quantity of the cocoon-forming insects to form a cocoon on the shelf (123);

d) transferring the shelf (123) comprising the cocoons to a second compartment in the second enclosure (201); and

e) waiting for at least some of the cocoon-forming insects that formed a cocoon to emerge from the cocoons.

In some embodiments, the method further comprises releasing the insects that emerge from cocoons out of the second compartment. In some embodiments, the first enclosure and the second enclosure are identical. In some embodiments, the first enclosure and the second enclosure are identical except the second enclosure has one or more extra corresponding sets of knock-in shelf supports knocked in. In illustrative embodiments, the first enclosure and the second enclosure are built from the same mold, with more corresponding sets of knock-in shelf supports knocked in in the second enclosure. In some embodiments, the second enclosure comprises one, two, or three shelves. In some embodiments, the second enclosure further comprises one or more middle compartments between the top compartment and the bottom compartment bounded by corresponding sets of knock-in shelf supports.

The foregoing description of illustrative embodiments of the invention has been presented for purposes of illustration and description, and is not intended to be exhaustive or to limit the invention to the precise form disclosed. The description was selected to best explain the principles of the invention and their practical application to enable others skilled in the art to best utilize the invention in various embodiments and various modifications as are suited to the particular use contemplated. It is intended that the scope of the invention not be limited by the specification, but be defined by the claims set forth below.

Those skilled in the art can devise many modifications and other embodiments within the scope and spirit of the present disclosure. Indeed, variations in the materials, methods, drawings, experiments, examples, and embodiments described may be made by skilled artisans without changing the fundamental aspects of the present disclosure. Any of the disclosed embodiments can be used in combination with any other disclosed embodiment.

In some instances, some concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

I claim:

1. An enclosure for raising cocoon-forming insects comprising:
a left side, a right side, a front, a back, a top, and a bottom;
the left side, the right side, and the front each comprising a screen;
the left side and the right side comprising at least one set of corresponding knock-in shelf supports dividing the interior of the enclosure into at least two compartments, the at least two compartments comprising a top compartment and a bottom compartment;
each set of corresponding knock-in shelf supports having, on the left side of the enclosure and the right side of the enclosure, similar distances from the bottom of the enclosure; and
the sets of corresponding knock-in shelf supports, when knocked-in on the left side and the right side, configured to support a shelf.

2. The enclosure of claim 1, wherein at least one set of corresponding knock-in shelf supports is knocked in on the left side and on the right side.

3. The enclosure of claim 2, further comprising a first shelf, resting on a first set of corresponding knocked-in shelf supports.

4. The enclosure of claim 3, wherein the first shelf is made of cardboard.

5. The enclosure of claim 3, wherein the enclosure comprises at least three sets of corresponding knock-in shelf supports.

6. The enclosure of claim 5, wherein at least three sets of corresponding knock-in shelf supports is knocked in on the left side and on the right side.

7. The enclosure of claim 6, further comprising a second shelf resting on a second set of corresponding knock-in shelf supports that have been knocked in and a third shelf resting on a third set of corresponding knock-in shelf supports that have been knocked in.

8. The enclosure of claim 7, wherein the first shelf, the second shelf, and the third shelf are made of cardboard.

9. The enclosure of claim 1, wherein the front is not removeable from the enclosure.

10. The enclosure of claim 1, wherein at least one compartment is accessible from the front and without accessing any other compartments by limiting the amount of creases the door is opened.

11. The enclosure of claim 1, wherein the enclosure is made of cardboard.

12. The enclosure of claim 1, wherein a hot adhesive is used to attach the left and right sides and the front of the screens to the enclosure.

13. The enclosure of claim 1, wherein glue is used to attach the left side, right side, front, back, top, and bottom of the enclosure to each other.

14. The enclosure of claim 3, wherein the first shelf is removeable from the enclosure.

15. The enclosure of claim 14, wherein the first shelf is located closer to the top of the enclosure than to the bottom of the enclosure.

16. The enclosure of claim 7, wherein the first shelf, the second shelf, and the third shelf are removeable from the enclosure.

17. The enclosure of claim 16, wherein the first shelf, the second shelf, and the third shelf can be rotated to the top of the enclosure.

18. The enclosure of claim 1, wherein the back comprises a screen.

19. A method of raising cocoon-forming insects, comprising the steps of:
a) placing a plurality of cocoon-forming insects in a bottom compartment of a first enclosure (101), wherein the plurality of cocoon-forming insects are at a life stage before forming a cocoon in a first compartment of the first enclosure;
b) feeding the plurality of cocoon-forming insects;
c) waiting for a quantity of the cocoon-forming insects to form a cocoon on the shelf (123);
d) transferring the shelf (123) comprising the cocoons to a second compartment in a second enclosure (201); and
e) waiting for at least some of the cocoon-forming insects that formed a cocoon to emerge from the cocoons.

20. A method of raising cocoon-forming insects in a first enclosure and a second enclosure, comprising the steps of:
a) placing a plurality of cocoon-forming insects in a bottom compartment of the first enclosure, wherein the plurality of cocoon-forming insects are at a life stage before forming a cocoon in a first compartment of the first enclosure;
the first enclosure comprising:
a first left side, a first right side, a first front, a first back, a first top, and a first bottom;
the first left side, the first right side, and the first front each comprising a screen;
the first left side and the first right side comprising at least one set of corresponding knock-in shelf supports dividing the interior of the first enclosure into at least two compartments, the at least two compartments comprising a top compartment and the bottom compartment;
each set of corresponding knock-in shelf supports having, on the first left side of the first enclosure and the first right side of the first enclosure, similar distances from the first bottom of the first enclosure;
the sets of corresponding knock-in shelf supports, when knocked-in on the first left side and the first right side, capable of supporting a shelf;
b) feeding the plurality of cocoon-forming insects;
c) waiting for a quantity of the cocoon-forming insects to form a cocoon on the shelf;
d) transferring the shelf comprising the cocoons to a second compartment in the second enclosure; and
e) waiting for at least some of the cocoon-forming insects that formed a cocoon to emerge from the cocoons.
